(19) United States
(12) Patent Application Publication
(10) Pub. No.: US 2011/0071500 A1
(43) Pub. Date: Mar. 24, 2011

(54) BRANCHED CATHETER TIP

(75) Inventor: Raymond LAREAU, Westford, MA (US)

(73) Assignee: NAVILYST MEDICAL, INC., Marlborrough, MA (US)

(21) Appl. No.: 12/563,621

(22) Filed: Sep. 21, 2009

(51) Int. Cl. A61M 25/00

(52) U.S. Cl. 604/523

(57) ABSTRACT

A venous access device comprises a catheter portion extending longitudinally from a proximal end to a distal end, the flexible shaft defining a lumen therein in combination with a plurality of branches separated from one another and extending distally from the flexible shaft.

### BACKGROUND

Venous access devices (VADs) include ports and indwelling catheters such as peripherally inserted central catheters (PICCs) that include flexible tubes used to supply fluids to internal body structures (e.g., nutrients, medications, blood products, etc.) over extended periods of time. Indwelling catheters may also be used to aspirate fluids such as blood from the patient for diagnostic procedures or to drain fluids for other therapeutic purposes. An indwelling catheter may, for example, include a proximal portion extending outside the skin or inside a pocket under the skin, while a distal tip extends through the body often along a tortuous path to enter a target body structure—e.g., the superior vena cava. To reduce trauma to tissue along this path, indwelling catheters are often made smaller in diameter, resulting in reduced flow rates. Additionally, the implanted portion of the catheter may be susceptible to fibrin sheath adherence and encrustation, which may reduce the mobility of the implanted portion.

### SUMMARY OF THE INVENTION

The present invention is directed to venous access devices that include branches at the distal end to enhance device flexibility and to minimize the risk of fibrin sheath adherence and encrustation by blood or other bodily fluids.

In one embodiment, the present invention is a venous access device that comprises a catheter portion extending longitudinally from a proximal end to a distal end. The catheter portion includes a first lumen extending in a proximal portion thereof. At its distal end, the device comprises a plurality of branches separated from one another and extending distally from the catheter proximal portion. Each of the plurality of branches includes a lumen that extends between the lumen that extends within the proximal portion of the catheter portion, and a distal end that opens to an exterior of the device.

In another embodiment, the present invention is a venous access device that comprises a multi-lumen catheter portion extending longitudinally from a proximal end to a distal end. The catheter portion includes a plurality of lumens, each extending in a proximal portion thereof. At its distal end, the device divides into sections, with each section containing at least one of the plurality of lumens, and each section further comprising a plurality of branches separated from one another and extending distally therefrom. Each of the plurality of branches includes a lumen that extends between the lumen(s) that extends within each section and a distal end that opens to an exterior of the device.

### BRIEF DESCRIPTION OF THE DRAWINGS

FIG. 1 shows a perspective view of a venous access device according to an exemplary embodiment of the present invention;

FIG. 2 shows a cross-sectional view of the system of FIG. 1, along line A-A.

FIG. 3 shows a cross-sectional view of the system of FIG. 1, along line B-B.

FIG. 4 shows a cross-sectional view of the system of FIG. 1, along line B-B, according town alternate embodiment of the present invention.

FIG. 5 shows a perspective view of a venous access device according to an exemplary embodiment of the present invention.

### DETAILED DESCRIPTION

The present invention may be further understood with reference to the following description and the appended drawings, wherein like elements are referred to with the same reference numerals. The present invention relates to devices for the transfer of fluids to and from a target structure within a patient's body over an extended period of time. In particular, exemplary embodiments of the present invention describe a venous access device that includes a branched distal tip for improved mobility, flexibility and flow.

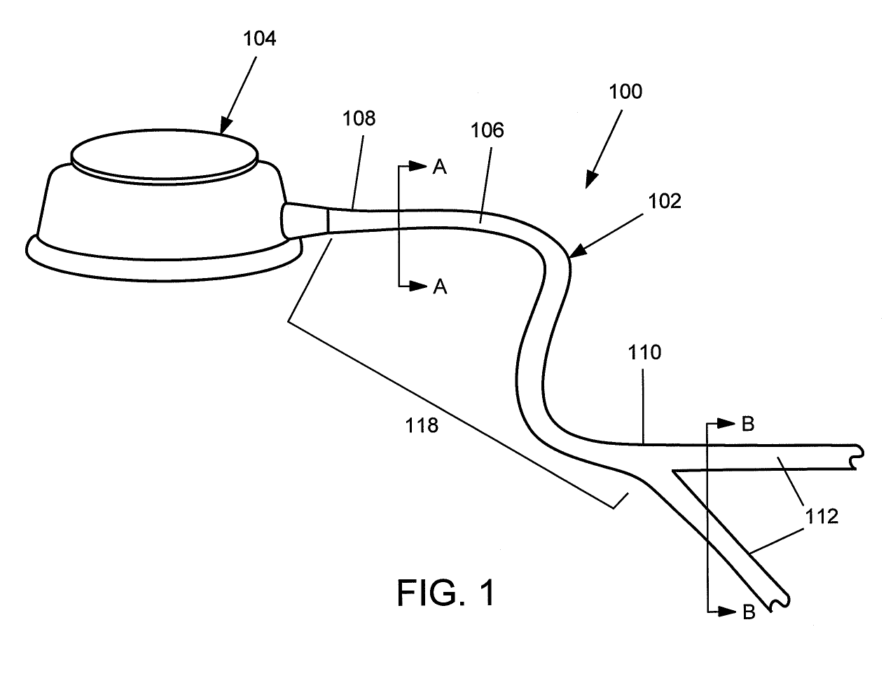

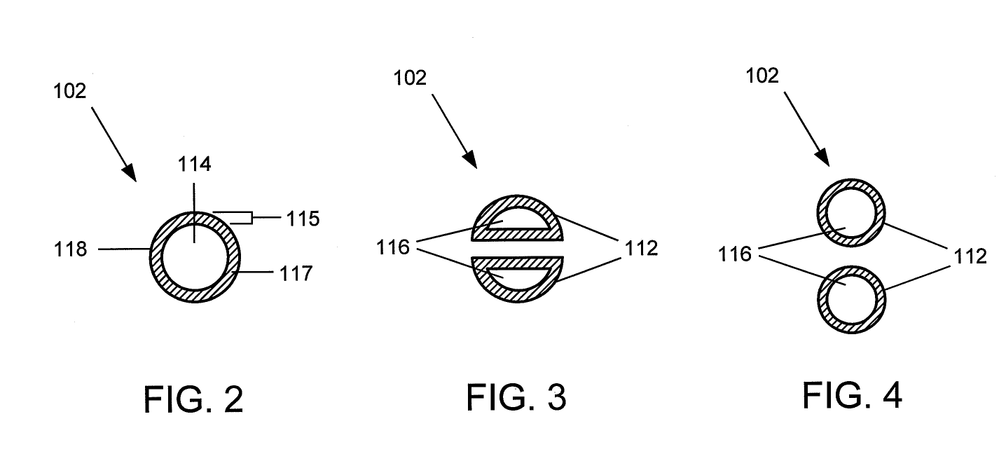

As shown in FIGS. 1-3, a venous access device 100 according to an exemplary embodiment of the present invention comprises an indwelling catheter portion 102 a proximal end 108 of which is connected to a port device 104. It will be understood by those of skill in the art that, although the device 100 is described as a port catheter system with a port device 104, the device 100 may be any venous access device, such as PICCs, central venous catheters (CVCs), dialysis catheters, and midline infusion catheters. For example, the catheter portion 102 may extend out of the skin to any known port connectable to external devices as desired and which may be sealed between uses such as, for example, a drainage bag for a drainage catheter system.

As shown in FIG. 1, the catheter portion 102 includes a shaft 106 extending from the proximal end 108 to a distal end 110 which, in an operative position, is inserted into a target structure within a patient's body. A proximal portion 118 of the shaft 106 includes a single first lumen 114 extending therethrough. A thickness 115 of a wall 117 of the shaft 106 around a circumference thereof may be selected as would be understood by those of skill in the art to achieve the desired properties (e.g., burst strength, flexibility, etc.) while the first lumen 114 is shaped to maximize its cross-sectional area without compromising these properties. At a distal end 110, the shaft 106 splits into a plurality of branches 112 at which the first lumen 114 opens to two lumens 116 each of which extends through a corresponding one of the branches 112. In a preferred embodiment, an outer diameter of each of the plurality of branches 112 is smaller than an outer diameter of the proximal portion 118 of the shaft 106. In alternate embodiments, each branch 112 has substantially the same diameter or greater diameters as compared to that of the proximal portion 118. In yet other embodiments, each branch 112 has a different size (e.g., length and/or diameter) from other branches, and/or a different cross sectional configuration (e.g., round, oval, trapezoid, D-shaped, etc.) from other branches. In a preferred embodiment, the shaft 106 splits into two branches 112, although it will be understood by those of skill in the art that the shaft 106 may split into any number of branches 112. FIG. 3 shows an embodiment of the catheter portion 102 in which the cross-section of lumens 116 are substantially D-shaped in the same manner as a lumen of a standard dual-lumen catheter. However, as shown in FIG. 4, either or both of the lumens 116 may be substantially round.

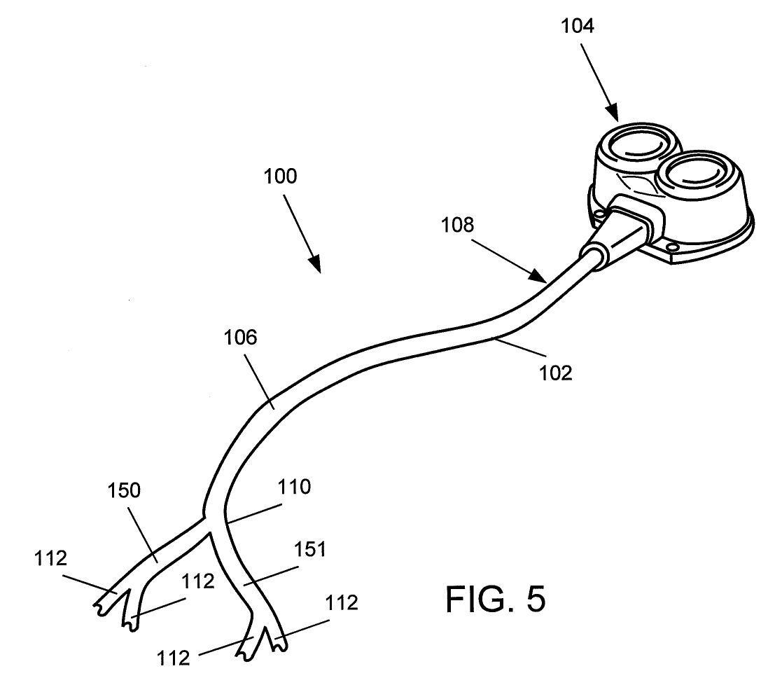

In another embodiment as shown in FIG. 5, the venous access device of the present invention includes a dual-lumen catheter portion 102. Such dual-lumen devices are known in the art, and include devices such as dual-lumen ports (as shown in FIG. 5), PICCs, and other implantable devices that include separate lumens that can be used to deliver different or the same materials such as therapeutic agents or contrast agents, or can be used to aspirate blood or other bodily fluids. In accordance with the present invention, the catheter portion 102 is divided into two sections 150, 151 at the distal end 110, with each section including one of the lumens that extend from the proximal end 108 to distal end 110. Each such section 150 is thereafter split into multiple branches 112 as described with reference to FIGS. 1-4, above.

The inventors have found that the use of branches 112 enhance the mobility and flexibility of the indwelling portion of the catheter portion 102 as compared to a single tube catheter. For example, in embodiments where the outer diameter of each of the branches 112 is smaller than the outer diameter of the proximal portion 118, it will be understood by those of skill in the art that the distal end 110 of the device is more flexible than the proximal portion 118. This enhanced flexibility allows for greater movement of the device 100 at its distal end 110 while implanted, such as with normal cardiovascular flow and pulse. The added flexibility and motion of the branches 112 inhibits fibrin sheath adherence and encrustation by blood or other bodily fluids, as sometimes encountered with single tube catheters.

In use, fluid flowing distally through the first lumen 114 splits into the lumens 116 as it leaves the proximal portion 118 and enters the distal end 110 of the device 100. In one embodiment, one or all of the lumens 116 taper toward the distal end 110 (i.e., a cross-sectional area of the lumen may grow gradually smaller from the proximal end of the corresponding branch 112 toward the distal end thereof). In addition, any or all of the branches 112 may be angled relative to one another, depending upon a desired performance aspect. That is, longitudinal axes of the branches 112 may be set at any desired angle relative to a longitudinal axis of a distal end of the proximal portion of the shaft 106 to achieve a desired orientation of the branches 112 relative to one another. Preferably, this angle is minimized to between 0 and 45 degrees, and more preferably between 0 and 15 degrees, such that the branches are in-line with the catheter longitudinal axis. As an example, the proximal portion 118 near the distal end 110 can define a first longitudinal axis and the first and second branches 112 can define second and third longitudinal axes, respectively. The a preferred embodiment, the second axis extends at an angle of less than 45 degrees from the first axis and the third axis is substantially co-axial with the first axis.

Those skilled in the art will understand that, for a catheter portion 102 including more than two branches 112, the various branches 112 may be located in a single plane or a plurality of planes. The length of each branch is preferably less than about eight centimeters from most venous access applications.

The distal end 110, including the branches 112 may be delivered to a target area of the body in the same manner used for conventional split tip catheters as will be understood by those skilled in the art. The branches 112 may be temporarily bonded to one another or held together by an insertion device to facilitate the passage of the catheter portion 102 through intervening tissue with a minimum of trauma thereto.

It will be understood by those skilled in the art that a catheter according to the present invention may include multiple lumens through a proximal part thereof so long as at least one of these proximal lumens opens to a plurality of lumens in separate distal branches of the catheter. That is, a catheter having a proximal part including two lumens will include at least three branches at a distal end thereof with each branch including a lumen open to one or more of the lumens of the proximal part.

It will be apparent to those skilled in the art that various modifications and variations can be made in the structure and methodology of the present invention. Thus, it is intended that the present invention cover the modifications and variations of this invention provided that they come within the scope of the appended claims and their equivalents.

What is claimed is:

1. A venous access device comprising:
a flexible shaft extending from a proximal end to a distal end, the flexible shaft having a first lumen therein;
a plurality of branches extending distally from the flexible shaft, a first one of the branches including a second lumen extending therethrough between a first branch proximal end open to the first lumen and a first branch distal end open to an exterior of the catheter, a second one of the branches including a third lumen extending therethrough between a second branch proximal end open to the first lumen and a second branch distal end open to an exterior of the catheter.

2. The venous access device of claim 1, wherein a cross-sectional area of each of the branches is less than a cross-sectional area of the flexible shaft.

3. The venous access device of claim 1, wherein a cross-sectional area of each of the branches is greater than a cross-sectional area of the flexible shaft.

4. The venous access device of claim 1, wherein a cross-sectional shape of the first lumen is different than a cross-sectional shape of the second lumen.

5. The venous access device of claim 4, wherein the first lumen is substantially circular in cross-section and the second lumen includes a planar first wall and a semi-circular second wall.

6. The venous access device of claim 1, wherein the first and second branches extend away from the distal end of the flexible shaft by an equal length.

7. The venous access device of claim 1, wherein the first branch is longer than the second branch.

8. The venous access device of claim 1, wherein the first, second and third lumens are substantially circular in cross-section.

9. The venous access device of claim 1, wherein the flexible shaft defines a first longitudinal axis and the first and second branches define second and third longitudinal axes, respectively, the second axis extending at an angle of between 0 degrees and 45 degrees relative to the first axis and the third axis substantially co-axial with the first axis.

10. The venous access device of claim 1, wherein the second axis extends at an angle of between 0 degrees and 10 degrees relative to the first axis and the third axis substantially co-axial with the first axis.

11. The venous access device of claim 1, wherein the angle between any two of said plurality of branches is less than about 15 degrees.

12. The venous access device of claim 1, wherein a total flow area of the second and third lumens is greater than a flow area of the first lumen.

13. A venous access device comprising:
a flexible shaft extending from a proximal end to a distal end, the flexible shaft having a plurality of shaft lumens therein;
a plurality of sections extending from the flexible shaft, each of the sections comprising at least one section lumen therein in fluid communication with at least one of the plurality of shaft lumens; and
a plurality of branches extending distally from each of the plurality of sections, each of the branches comprising a branch lumen therein in communication with at least one section lumen.
